(19) United States
(12) Patent Application Publication
(10) Pub. No.: US 2016/0354057 A1
(43) Pub. Date: Dec. 8, 2016

(54) ULTRASOUND IMAGING SYSTEM AND ULTRASOUND-BASED METHOD FOR GUIDING A CATHETER

(71) Applicant: General Electric Company, Schenectady, NY (US)

(72) Inventors: Gunnar Hansen, Horten (NO); Olivier Gerard, Horten (NO)

(21) Appl. No.: 14/733,537

(22) Filed: Jun. 8, 2015

(51) Int. Cl. A61B 8/08; A61B 8/00; A61B 90/00; A61B 5/06

(52) U.S. Cl. CPC A61B 8/0841; A61B 5/062; A61B 8/463; A61B 8/483; A61B 90/37; A61B 2090/378

(57) ABSTRACT

An ultrasound imaging system and an ultrasound-based method for guiding a catheter during an interventional procedure include acquiring 3D ultrasound data, identifying a reference location, displaying an ultrasound image based on the 3D ultrasound data, and displaying a guideline superimposed on the ultrasound image, where the guideline represents the intended insertion path for the catheter with respect to the reference location.

### FIELD OF THE INVENTION

This disclosure relates generally to an ultrasound imaging system and an ultrasound-based method for guiding a catheter during an interventional procedure.

### BACKGROUND OF THE INVENTION

In order for an implantable medical device to have maximum efficacy with minimal risk for a given clinical indication, it is critical to guide and position the implantable medical device as accurately as possible. According to conventional techniques, many implantable medical devices are inserted via a catheter and guided with a 2D fluoroscopic X-ray image showing the real-time progress of the catheter through the patient's body. While the 2D fluoroscopic X-ray image advantageously shows the position of the catheter in real-time, there are several disadvantages associated with relying primarily on a 2D fluoroscopic X-ray image for the guidance and ultimate placement of the implantable medical device in 3D space.

First, because the 2D fluoroscopic X-ray image is a 2D image from a single view direction, it is only possible to tell how the catheter and the medical device are positioned with respect to the plane of the 2D image. In other words, it is difficult or impossible to tell how the catheter and medical device are positioned in directions that are “out-of-plane.” For example, if the 2D image represents an x-y plane, it is difficult or impossible to tell, based solely on a 2D image, how the catheter is positioned with respect to a z-direction perpendicular to the x-y plane.

Second, a 2D fluoroscopic X-ray image shows the X-ray attenuation of the tissue being examined. Dense X-ray attenuating structures and materials, such as bones, catheters, and medical devices, are typically very clearly visible in a 2D fluoroscopic X-ray image. However, X-rays are not as useful for imaging soft tissue. Therefore, when relying on a 2D fluoroscopic X-ray image to guide a catheter and a medical device, the clinician does not have the benefit of detailed real-time information about the relative positioning of the catheter and medical device with respect to soft tissue structures within the patient. For example, when the implantable medical device is a valve and the procedure includes replacing a mitral valve or an aortic valve, improper positioning of the implantable medical device (valve) may result in embolization of the device, coronary obstruction, or a paravalvular leak.

Third, relying on a 2D fluoroscopic X-ray image exposes both the patient and the clinician to X-ray dose the entire time the X-ray tube is turned on and emitting X-rays. There is increasing concern regarding exposure to X-ray dose, and it would be beneficial to develop procedures that result in less overall dose for both the patient and clinician.

For these and other reasons, an improved ultrasound imaging system and an ultrasound-based method for guiding a catheter during an interventional procedure are desired.

### BRIEF DESCRIPTION OF THE INVENTION

The above-mentioned shortcomings, disadvantages, and problems are addressed herein which will be understood by reading and understanding the following specification.

In an embodiment, an ultrasound-based method for guiding a catheter during an interventional procedure comprises acquiring 3D ultrasound data, identifying a reference location based on the 3D ultrasound data, displaying an ultrasound image based on the 3D ultrasound data, displaying a guideline superimposed on the ultrasound image, where the guideline represents an intended insertion path for the catheter with respect to the reference location, inserting the catheter during the process of both acquiring the 3D ultrasound data and displaying the guideline superimposed on the ultrasound image.

In an embodiment, an ultrasound imaging system includes a probe, a display device, and a processor in electronic communication with the probe and the display device. The processor is configured to control the probe to acquire 3D ultrasound data, display an ultrasound image based on the 3D ultrasound data on the display device, identify a reference location in the 3D ultrasound data, display a guideline superimposed on the ultrasound image, where the guideline represents an intended insertion path for a catheter with respect to the reference location, automatically detect a position of the catheter based on the 3D ultrasound data, and automatically provide feedback indicating whether the catheter is within a predetermined distance from the intended insertion path during the process of inserting the catheter.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a flow chart of a method in accordance with an exemplary embodiment;

FIG. 3 is a schematic representation of an image according to an exemplary embodiment;

FIG. 4 is a schematic representation of a display in accordance with an exemplary embodiment;

FIG. 5 is a schematic representation of a display in accordance with an exemplary embodiment; and

FIG. 6 is a schematic representation of an image of a heart in accordance with an exemplary embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical, and other changes may be made without departing from the scope of the embodiments. The following detailed description is, therefore, not to be taken as limiting the scope of the invention.

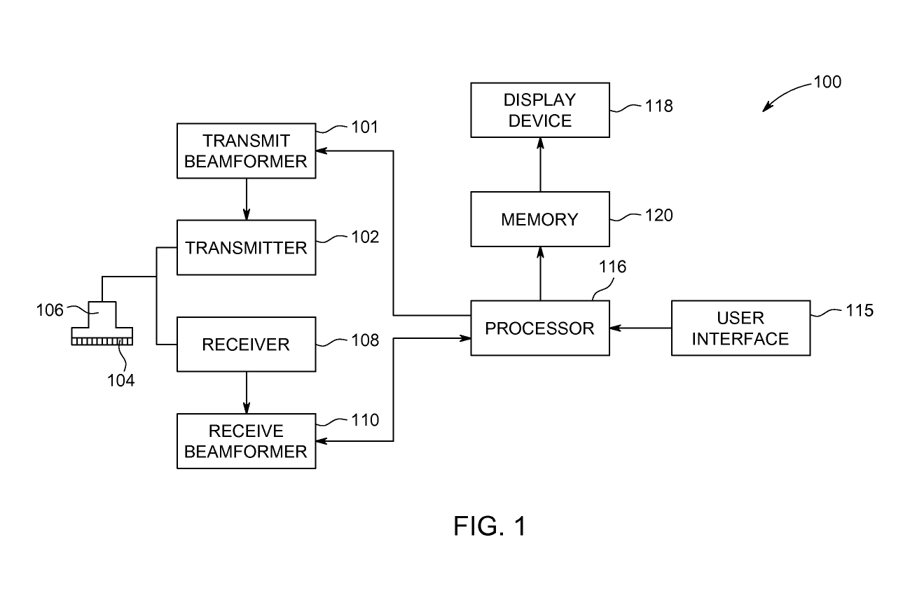

FIG. 1 is a schematic diagram of an ultrasound imaging system 100. The ultrasound imaging system 100 includes a transmit beamformer 101 and a transmitter 102 that drive elements 104 within a probe 106 to emit pulsed ultrasonic signals into a body (not shown). According to an embodiment, the probe 106 may be capable of acquiring real-time 3D ultrasound images. For example, the probe 106 may be a mechanical probe that sweeps or oscillates an array in order to acquire the real-time 3D ultrasound data, or the probe 106 may be a 2D matrix array with full beam-steering in both the azimuth and elevation directions. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104, and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface 115 may be used to control the input of patient data, or to select various modes, operations, and parameters, and the like. The user interface 115 may include a one or more user input devices such as a keyboard, hard keys, a touch pad, a touch screen, a track ball, rotary controls, sliders, soft keys, or any other user input devices.

The ultrasound imaging system 100 also includes a processor 116 to control the transmit beamformer 101, the transmitter 102, the receiver 108, and the receive beamformer 110. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The receive beamformer 110 may be configured to perform conventional beamforming techniques as well as techniques such as retrospective transmit beamforming (RTB).

The processor 116 is in electronic communication with the probe 106. The processor 116 may control the probe 106 to acquire ultrasound data. The processor 116 controls which of the elements 104 are active and the shape of a beam emitted from the probe 106. The processor 116 is also in electronic communication with a display device 118, and the processor 116 may process the ultrasound data into images for display on the display device 118. For purposes of this disclosure, the term “electronic communication” may be defined to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU), or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation may be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. Real-time volume rates may vary based on the size of the volume from which data is acquired and the specific parameters used during the acquisition. The data may be stored temporarily in a buffer during a scanning session and processed in less than real-time in a live or off-line operation. Some embodiments of the invention may include multiple processors (not shown) to handle the processing tasks. For example, an embodiment may use a first processor to demodulate and decimate the RF signal and a second processor to further process the data prior to displaying an image. It should be appreciated that other embodiments may use a different arrangement of processors. For embodiments where the receive beamformer 110 is a software beamformer, the processing functions attributed to the processor 116 and the software beamformer hereinabove may be performed by a single processor, such as the receive beamformer 110, or the processor 116. Or the processing functions attributed to the processor 116 and the software beamformer may be allocated in a different manner between any number of separate processing components.

According to an embodiment, the ultrasound imaging system 100 may continuously acquire real-time 3D ultrasound data at a volume-rate of, for example, 10 Hz to 30 Hz. A live ultrasound image may be generated based on the real-time 3D ultrasound data. The live ultrasound image may be refreshed at a frame-rate that is similar to the volume-rate according to an embodiment. Other embodiments may acquire data and or display the live ultrasound image at different volume-rates and/or frame-rates. For example, some embodiments may acquire real-time 3D ultrasound data at a frame-rate of less than 10 Hz or greater than 30 Hz depending on the size of the volume and the intended application. Other embodiments may use 3D ultrasound data that is not real-time 3D ultrasound data. A memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may comprise any known data storage medium. In embodiments where the 3D ultrasound data is not real-time 3D ultrasound data, the 3D ultrasound data may be accessed from the memory 120, or any other memory or storage device. The memory or storage device may be a component of the ultrasound imaging system 100, or the memory or storage device may external to the ultrasound imaging system 100.

Optionally, embodiments of the present invention may be implemented utilizing contrast agents and contrast imaging. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component, and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate and combinations thereof, and the like) to form 2D or 3D images or data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from beam space coordinates to display space coordinates. A video processor module may be provided that reads the image frames from a memory and displays the image frames in real time while a procedure is being carried out on a patient. A video processor module may store the image frames in an image memory, from which the images are read and displayed.

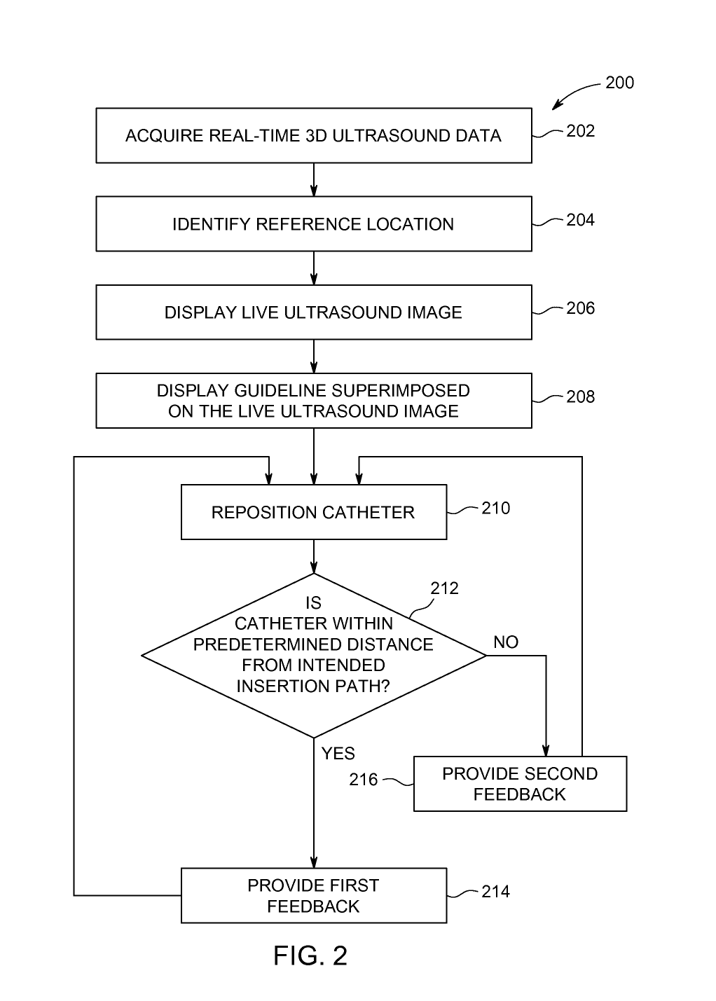

FIG. 2 is a flow chart of a method in accordance with an exemplary embodiment. The individual blocks of the flow chart represent steps that may be performed in accordance with the method 200. Additional embodiments may perform the steps shown in a different sequence, and/or additional embodiments may include additional steps not shown in FIG. 2. The technical effect of the method 200 is the displaying of a guideline representing an intended insertion path on a live ultrasound image and providing feedback regarding whether or not a catheter is within a predetermined distance from the intended insertion path during the process of inserting the catheter.

At step 202, the processor 116 controls the transmit beamformer 101, the transmitter 102, the probe 106, the receiver 108, and the receive beamformer 110 to acquire real-time 3D ultrasound data from a volume-of-interest. For purposes of this disclosure, the term “real-time 3D ultrasound data” is defined to include ultrasound data that includes a plurality of volumes acquired from a volume-of-interest. Each volume of ultrasound data may represent the volume-of-interest at a different point in time. As described with respect to FIG. 1, acquiring the real-time 3D ultrasound data may include acquiring ultrasound data, beamforming the ultrasound data with the receive beamformer 110, and then scan-converting the beamformed ultrasound data for display as a 3D ultrasound image.

At step 204, a reference location is identified based on the real-time 3D ultrasound data. The processor 116 may identify the reference location in an ultrasound image generated based on the real-time 3D ultrasound data. The ultrasound image may include a plane or a slice, or the image may include a 3D image, such as a volume-rendered image. The reference location may be identified through manual, automatic, or semi-automatic techniques. Hereinafter, the method 200 will be described according to an exemplary technique where the method 200 is used in a TAVI (Transcatheter Aortic Valve Implantation) procedure in order to replace an aortic valve. However, it should be appreciated that the TAVI procedure is just one exemplary procedure and that the method 200 may be used with many other types of procedures as well, including mitral valve replacement, left atrial appendage closure, and transseptal puncture. Those skilled in the art should appreciate that the method 200 may be used to perform other types of interventional procedures as well.

Manually identifying the reference location may include manually identifying a plurality of points or a contour associated with a particular structure. The points and/or contour may be identified on one or more frames of the live ultrasound image. According to an exemplary embodiment, the points and/or contour may be identified on a single frame of the live ultrasound image. For example, a clinician may freeze the live ultrasound image so as to view only a single frame instead of the live display of a sequence of frames. In the exemplary embodiment where the method 200 is used to perform a TAVI procedure, the reference location may include a plane of the aortic valve. The clinician may, for instance, identify a plurality of points on either a 3D image, such as a volume-rendered image, or on an image or slice derived from the 3D ultrasound data.

The reference location may also be automatically identified by the processor 116. For example, the processor 116 may implement an image processing technique in order to automatically identify the reference location. For example, the processor 116 may implement a border-detection algorithm to detect the anatomical structure. The border-detection algorithm may use a combination of techniques including a thresholding operation or a gradient detection operation. The processor 116 may either use the anatomical structure as the reference location, or the processor may determine the position of the reference location based on the detected anatomical structure. The processor 116 may implement image processing techniques on an image generated from 3D ultrasound data, or the processor 116 may implement the image processing techniques directly on the 3D ultrasound data.

Next, at step 206, the processor 116 displays a live ultrasound image based on the real-time 3D ultrasound data on the display device 118. For purposes of this disclosure, a live ultrasound image is defined to include a sequence of images based on real-time ultrasound data. Each image in the sequence represents data acquired during a different period of time. A live ultrasound image may include either a slice or a plane generated from the 3D ultrasound data, or the live ultrasound image may include a 3D ultrasound image, such as a volume-rendered image. A live ultrasound image of a slice or plane would show how the data along that particular slice or plane changes over time, while a live 3D ultrasound image would show how data from a particular volume-of-interest changes over time.

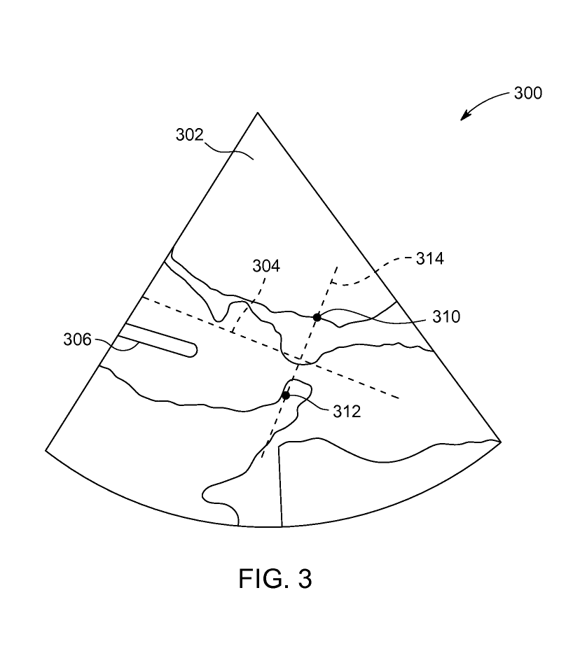

FIG. 3 is a schematic representation of image 300 according to an exemplary embodiment. The image 300 includes an ultrasound image 302, a guideline 304, a first point 310, a second point 312, an aortic valve plane 314, and a catheter 306. It should be appreciated that the aortic valve plane 314 is just one example of a reference location and that other reference locations may be used according to other embodiments. The catheter 306 is a portion of the ultrasound image 302 representing a catheter inserted into a patient's body. According to an embodiment, the ultrasound image 302 may be an image frame of the live ultrasound image displayed at step 206. While the image 300 represents a single frame, it should be appreciated that the image 300 and the position of the guideline 304 may be updated as additional ultrasound data is acquired and additional ultrasound image frames are generated from the real-time 3D ultrasound data. The aortic valve plane 314, the first point 310, and the second point 312 may not be displayed according to other embodiments.

At step 208, the processor 116 displays a guideline, such as the guideline 304, on the live ultrasound image. The guideline 304 is shown as a dashed line, but other embodiments may use guidelines that include solid lines, dotted lines, or multiple lines in order to specify the intended insertion path for the catheter. The user may control the path of the catheter in the patient's body by comparing the position and path of catheter 306 to the guideline 304. For example, multiple guidelines may be used to show an acceptable range with respect to catheter 306. According to an embodiment, the processor 116 may calculate the position of the guideline 304 in real-time as the real-time 3D ultrasound data is acquired. In the exemplary embodiment where the method 200 is used to perform a TAVI procedure, the processor 116 may determine the intended insertion path for the catheter with respect to a reference location. As described previously, the reference location may be the aortic valve plane 314. In the TAVI procedure, it is generally desirable to insert the catheter along a path that is perpendicular or generally perpendicular, such as within 5 or 10 degrees of perpendicular, to the aortic valve plane 314. The processor 116 may therefore calculate an intended insertion path that is positioned generally perpendicular to the aortic valve plane 314. The image 300 is a 2D image. However, it should be appreciated that the position of the reference location, such as the aortic valve plane 314, and the guideline 304 may be determined based on 3D ultrasound data.

It may be beneficial to identify one or more additional reference locations or structures in order for the processor 116 to more precisely determine the position for the guideline 304. For example, in a transapical approach, the guideline needs to enter the heart at the left ventricular apex and then approach the aortic valve plane so that the guideline (and intended insertion path) intersects the aortic valve plane in approximately the center of the existing aortic valve. As such, another reference location such as the left ventricular apex or another cardiac structure may be identified by manual, automatic, or semi-automatic techniques. The processor 116 may use any of these additional reference locations in order to more precisely position the guideline 304.

In a transfemoral approach, the catheter 122 is inserted through the aortic root. While it is still desirable to position the guideline so that it is perpendicular or generally perpendicular to the aortic valve plane, it is also desirable for the guideline to be positioned so that it is generally in the center of the aortic root and aligned with a long axis of the aortic root. Accordingly, the processor 116 may automatically identify the aortic root by image processing techniques such as a shape-based detection algorithm, fitting a deformable mesh to the real-time 3D ultrasound data, or any other image processing technique. Additionally, semi-automated or manual techniques may also be used. For example, a clinician may position one or more points on the edge of the aortic root, or the clinician may identify a contour defining the edge of the aortic root. According to an embodiment, the processor 116 may use these points or the contour to segment the aortic root and/or fit a deformable mesh to the 3D ultrasound data to track the position and orientation of the aortic root in real-time as additional 3D ultrasound data is acquired.

According to an exemplary embodiment, the processor 116 may automatically track the positions of one or more reference locations, such as the aortic valve plane 314 and the aortic root, in real-time as the ultrasound imaging system is acquiring real-time 3D ultrasound data. The processor 116 may therefore calculate the position of the guideline 304 (and, hence, the intended insertion path) based on the real-time positions and orientations of the reference locations. For example, the processor 116 may keep the guideline 304 in a fixed relative position with respect to the reference location 314 even while the patient's anatomy is in motion. During a cardiac interventional procedure, the patient's heart is constantly moving. In addition to normal cardiac function, there is always the possibility that the positions of the reference locations may be moved slightly as the catheter and implantable device are advanced into the patient. However, by tracking the position or positions of one or more reference locations, the processor 116 may adjust the positioning of the guideline 304 with respect to the reference locations in real-time to ensure that the clinician is following the most accurate path given the current, real-time position of the patient's anatomical structures. This technique positions the guideline 304 using the most up-to-date ultrasound information possible and, therefore, provides for increased patient safety and improved odds of a successful clinical outcome from the interventional procedure.

At step 210, the clinician repositions the catheter with respect to a patient while the ultrasound imaging system 100 continues to acquire real-time 3D ultrasound data and to display the guideline 304 on the ultrasound image. According to an embodiment, repositioning the catheter may include inserting the catheter into a patient. According to some embodiments, such as the exemplary embodiment where the method 200 is used to perform a TAVI procedure, the catheter may be used to insert and position a medical device, such as a replacement valve or any other type of medical device that may be inserted with a catheter. As described hereinabove, the processor 116 controls the ultrasound imaging system 100 to continue acquiring real-time 3D ultrasound data and to generate a live ultrasound image during the process of inserting the catheter into the patient. This allows the processor 116 to update the position of the guideline 304 in real-time based on the current position of one or more reference locations identified in the patient. Additionally, since ultrasound data is being used, the reference locations may be based on anatomical structures in soft tissue. For cardiac procedures, this is a significant advantage compared to conventional techniques relying on fluoroscopic images. Fluoroscopic images, acquired with X-rays, are not well-suited for displaying and tracking reference locations based on anatomical structures in soft tissue.

According to an embodiment, the processor 116 may be configured to automatically detect the position and orientation of the catheter in 3D space in real-time based on the real-time 3D ultrasound data or a live ultrasound image generated based on the real-time 3D ultrasound data. According to an embodiment, a tracking system, such as an electromagnetic tracking system, may be used to track the catheter's position. For example, an electromagnetic tracking device may be attached to the catheter and used to determine the catheter's position with respect to a known magnetic field. The processor 116 may then use data obtained from the electromagnetic tracking device to calculate whether or not the catheter is within the predetermined acceptable distance of the intended insertion path during the process of inserting the catheter and/or a medical device via the catheter.

According to other embodiments, image processing techniques may be used to detect the position of the catheter 306 in real-time based on the live image generated from the 3D real-time ultrasound data. In order to improve the speed and accuracy of the image processing techniques used to detect the catheter 306, the processor 116 may search for the catheter 306 only within a portion of the ultrasound image where the catheter 306 is expected. For example, in TAVI with a transfemoral approach, the processor may search for the catheter 306 only within the volume corresponding to the aortic root. As described above, according to an embodiment, the aortic root may have been previously identified and segmented during step 204. The processor 116 may, for instance, implement an edge-detection algorithm within the specified volume, such as the volume corresponding to the aortic root, in order to identify the catheter 306. In other embodiments, the processor 116 may search for the catheter 306 within a different volume. For example, the processor 116 may search for the catheter 306 by searching within a predetermined radius from the guideline 304 based on the assumption that the catheter 306 should be relatively near to the guideline 304. The processor 116 may also search for the catheter 306 by starting at the guideline 304 and searching in an ever-expanding radial direction (i.e., searching in a volume defined by a cylinder centered about the guideline 304, where a radius of the cylinder is increased until the catheter 306 is detected). According to still another embodiment, once the algorithm detects the catheter 306, the processor may use a priori information to limit the volume from which the catheter 306 is searched. For example, after detecting the catheter 306, the processor 116 may only search for the catheter 306 within a predetermined volume using the previously calculated catheter position to make an assumption about the most likely volume to contain the catheter 306. For example, the algorithm may start searching based on the most recently detected edge of the catheter 306 and work radially outward from the edge. The algorithm may also identify the tip of the catheter 306, and the processor 116 may display a graphical indicator on the live ultrasound image indicating the tip of the catheter 306. According to yet other embodiments, the processor 116 may search for the catheter 306 within a volume corresponding to a different anatomical structure or a volume defined in relationship to one or more different anatomical locations. The processor 116 may use image processing techniques to search for the catheter either in images generated from the 3D ultrasound data or directly from the 3D ultrasound data. For example, when searching for the catheter in a volume, the processor may implement image processing techniques on a volume-rendered image or directly from the 3D data.

After identifying an edge of the catheter 306, the processor 116 may then calculate a line based on the results of the edge detection and compare the position and orientation of the calculated line (representing the position of the catheter 306) with the position and orientation of the guideline 304 (representing the intended insertion path). The catheter 306 is typically easily visible to the clinician in the live ultrasound image based on the 3D real-time ultrasound data. However, some embodiments may display a line representing the real-time position and orientation of the catheter 306 on the live image. For example, a trajectory line, based on the current position and orientation of the catheter 306 may be displayed so that the clinician may more easily see any differences between the current position and orientation of the catheter 306 and the guideline 304 representing the intended insertion path. The catheter 306 may be colorized or otherwise enhanced so that it is more clearly visible in the live ultrasound image.

At step 212, the processor 116 may calculate whether or not the catheter is within a predetermined distance from the intended insertion path indicated by the guideline 304. The processor 116 may make the determination based on the detected position of the catheter 306, the orientation of the catheter 306, or a combination of the position and the orientation of the catheter 306. For example, the processor 116 may determine the position and orientation of a catheter line (not shown). The catheter line may, for example, be positioned along a longitudinal axis of the catheter 306, and it may represent the position and orientation of the catheter 306. The processor 116 may then compare the catheter line to the guideline 304 in multiple different cut-planes. The processor 116 may determine that the catheter 306 is within the predetermined distance from the guideline 304 based on whether or not the catheter 306 is within a predetermined number of degrees of offset in each of the multiple different cut-planes, for example. The processor 116 may optionally display a slice or cut-plane including both the catheter 306 and the guideline 304 to show how far the catheter in the patient is from the intended insertion path. According to other embodiments, the processor 116 may also determine if the catheter 306 is within the predetermined distance from the guideline 304 based on information regarding the position of a tip of the catheter 306. The processor 116 may also calculate a trajectory for the catheter 306 based on the real-time position and orientation of the catheter 306. The processor 116 may provide feedback regarding the trajectory of the catheter 306 according to an embodiment.

The processor 116 provides first feedback if the catheter is within a predetermined distance of the intended insertion path and second feedback if the catheter is outside of the predetermined distance of the intended insertion path. For example, at step 212, if the catheter 306 is within the predetermined distance from guideline 304, the method 200 advances to step 214, and the processor 116 provides first feedback. If, at step 212, the catheter 306 is not within the predetermined distance from the intended insertion path, the method advances to step 216, and the processor 116 provides second feedback. After providing either the first feedback at step 214 or the second feedback at step 216, the method 200 may return to step 210 and the position of the catheter 306 may be adjusted. This results in an updated position of the catheter with respect to the intended insertion path. Then, at step 212, the processor 116 may recalculate whether or not the catheter 306 is within the predetermined distance from the guideline 304 based updated position of the catheter 306. In some embodiments, guidelines may be displayed on the live image to visually indicate the range of the predetermined distance from the intended insertion path. If the catheter is within the predetermined distance from the intended insertion path, the processor 116 may control the ultrasound imaging system 100 to provide first feedback to the clinician. The first feedback may be visual, audible, or haptic. More information about the first feedback will be provided hereinafter.

The processor 116 provides second feedback if the catheter is outside of the predetermined distance from the intended insertion path. The second feedback may be visual, audible, or haptic. It is intended that the processor 116 will provide first feedback and second feedback to the clinician in real-time as the catheter is being inserted into the patient. Some exemplary types of feedback will be discussed hereinbelow.

According to an embodiment, the first feedback and/or the second feedback may include audible feedback. For example, the processor 116 may control a driver to generate a first tone or other type of audible feedback through a speaker if the catheter is within the predetermined distance from the intended insertion path (e.g., the first feedback may be the first tone). The processor 116 may control a driver to generate a second tone or other type of audible feedback through a speaker if the catheter is outside of the predetermined distance from the intended insertion path (e.g., the second feedback may be the second tone). Then, as the clinician is inserting the catheter, the tone played through the speaker will inform the clinician whether or not the catheter is within a predetermined distance from the intended insertion path or outside the predetermined distance from the intended insertion path. In other embodiments, the audible feedback may include a warning message played through a speaker when the catheter is outside of the predetermined distance from the intended insertion path. The feedback may also include a recorded message stating a word or a warning when the catheter is outside of the predetermined distance from the intended insertion path.

In other embodiments, the first feedback and/or the second feedback may include visual feedback. For example, the colorization of elements displayed on the display device 118, such as the catheter 306 or the guideline 304, may be adjusted to indicate whether the catheter is within the predetermined distance from the intended insertion path or outside of the predetermined distance from the intended insertion path. For example, the catheter 306 and/or the guideline 304 may be displayed in a first color to indicate that catheter 306 is within the predetermined distance from the guideline 304. The catheter 306 and/or the guideline 304 may be displayed in a second color to indicate that the catheter is outside of the predetermined distance from the guideline 304. In some embodiments, the visual feedback may include a visual warning when the catheter is outside of the predetermined distance from the intended insertion path. For example, the color of the image or a portion of the image may be adjusted, or a text-based warning message may be displayed on image. It should be appreciated by those skilled in the art than other uses of color, flashing, and text-based messages may be used to provide feedback to the user regarding whether the catheter is within the predetermined distance from the intended insertion path or outside of the predetermined distance from the intended insertion path. Some embodiments may only display visual feedback if the catheter is within the predetermined distance from the intended insertion path while other embodiments may only display visual feedback if the catheter is outside of the predetermined distance from the intended insertion path. Other embodiments may show visual feedback to indicate both if the catheter is within the predetermined distance and if the catheter is outside of the predetermined distance.

It should be appreciated by those skilled in the art that other techniques may be used to provide feedback regarding whether the catheter is within the predetermined distance from the intended insertion path or whether the catheter is outside of the predetermined distance from the intended insertion path. For example, haptic feedback, including vibration, may be used. Additionally, an embodiment may use more than one type of feedback to indicate whether the catheter is within the predetermined distance from the intended insertion path or outside of the predetermined distance from the intended insertion path. For example, two or more different types of feedback selected from a group including audible, visual, and haptic may be used to help inform the clinician while during the process of inserting the catheter.

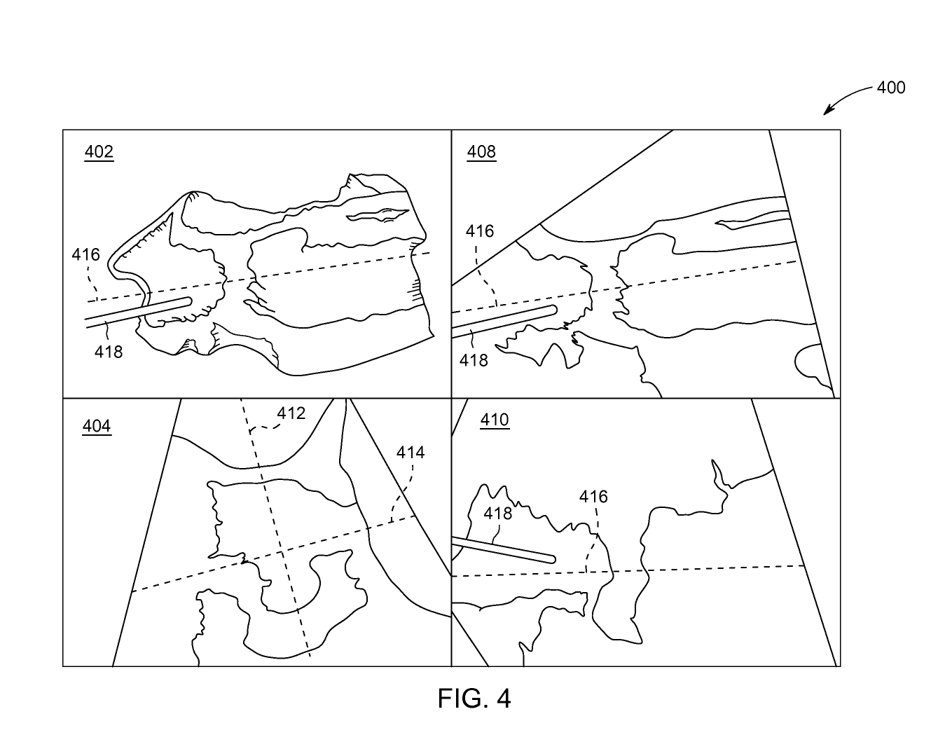

FIG. 4 is a schematic representation of a display 400 from a display device such as the display device 118 shown in FIG. 1 in accordance with an exemplary embodiment. The display 400 includes a volume-rendered image 402, a short-axis image 404, a first long-axis image 408, and a second long-axis image 410. The volume-rendered image 402, the short-axis image 404, the first long-axis image 408, and the second long-axis image 410 may all be generated from real-time 3D ultrasound data. The short-axis image 404, the first long-axis image 408, and the second long-axis image 410 each represents an image of a plane intersecting the structure shown in the volume-rendered image 402. The relative positions of the first long-axis image 408 and the second long-axis image 410 may be determined based on the short-axis image 404. For example, the short-axis image 404 includes a first dashed line 412 and a second dashed line 414. According to the exemplary embodiment shown in FIG. 4, the short-axis image 404 represents a plane that is perpendicular to the first long-axis image 408 and the second long-axis image 410. The first dashed line 412 shows the position of the first plane represented in the first long-axis image 408 with respect to the short-axis image 404. The second dashed line 414 shows the position of the second plane represented in the second long-axis image 410. The first dashed line 412 may be a first color, and the second dashed line 414 may be a second color. The first color and the second color may be used to associate the first dashed line 412 with the first long-axis image 408 and the second dashed line 414 with the second long-axis image 410. For example, a portion of the first long-axis image 408, such as a border around the first long-axis image 408, may be shown in the first color. Likewise, a portion of the second long-axis image 410, such as a border around the second long-axis image, may be shown in the second color. This way it is easy for the user to quickly understand that the first dashed line 412 corresponds to the first long-axis image 408 and that the second dashed line 414 corresponds to the second long-axis image 410.

A guideline 416 and a catheter 418 are shown in the volume-rendered image 402, the first long-axis image 408, and the second long-axis image 410. The user is able to clearly comprehend the precise position of the catheter with respect to the intended insertion path by referencing the catheter 418, the volume-rendered image 402, the first long-axis image 408, and the second long-axis image 410.

According to an embodiment, the user may adjust the position of the first long-axis image 408 and the second long-axis image 410. For example, the user may select either the first dashed line 412 or the second dashed line 414 in the short-axis image 404 and manipulate the position of the selected dashed line with respect to the short-axis image 404 in order to adjust the position of the corresponding long-axis image. For example, by selecting the first dashed line 412, the user is able to easily adjust the plane represented in the first long-axis image 408 by manipulating the position of the first dashed line 412. Or, by selecting the second dashed line 414, the user is able to easily adjust the plane represented in the second long-axis image 410 by manipulating the position of the second dashed line 414.

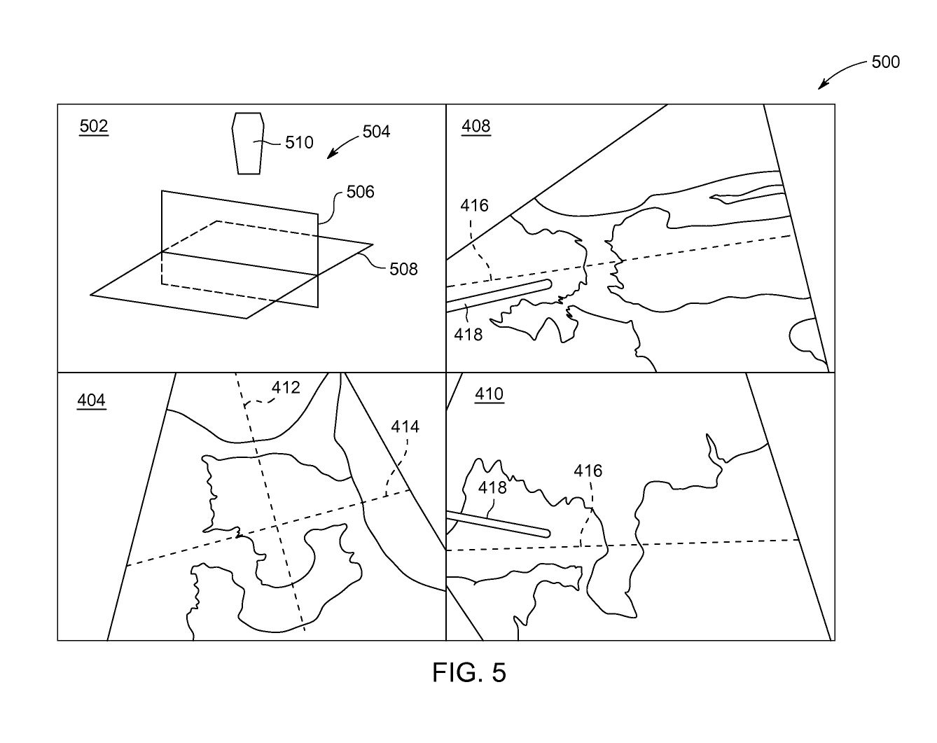

FIG. 5 is a schematic representation of a display 500 in accordance with an embodiment. FIG. 5 includes elements that are identical to elements previously described with respect to FIG. 4. Common reference numbers are used to identify identical elements in both FIGS. 4 and 5. Elements that were previously described with respect to FIG. 4 will not be described in detail with respect to FIG. 5.

The display 500 includes four images: a first image 502, the short-axis image 404, the first long-axis image 408, and the second long-axis image 410. The short-axis image 404, the first long-axis image 408, and the second long-axis image 410 are identical to the identically named elements previously described with respect to FIG. 4. The first image 502 includes a navigational icon 504. The navigational icon 504 includes a first plane 506 and a second plane 508 shown with respect to a probe model 510. The position of the first plane 506 with respect to the probe model 510 indicates the position of the plane represented by the first long-axis image 408. The position of the second plane 508 with respect to the probe model 510 indicates the position of the plane represented by the second long-axis image 410. Additionally, the first plane 506 corresponds with the first dashed line 412, and the second plane 508 corresponds with the second dashed line 414.

It should be appreciated that FIGS. 4 and 5 represent two exemplary embodiments of displays that may be used to display images and that displays may show either more than, or fewer than, four images at a time according to other embodiments. Additionally, the images may represent different planes, and/or the planes represented by the images may have different relative orientations than those shown in either FIG. 4 or FIG. 5 according to other embodiments.

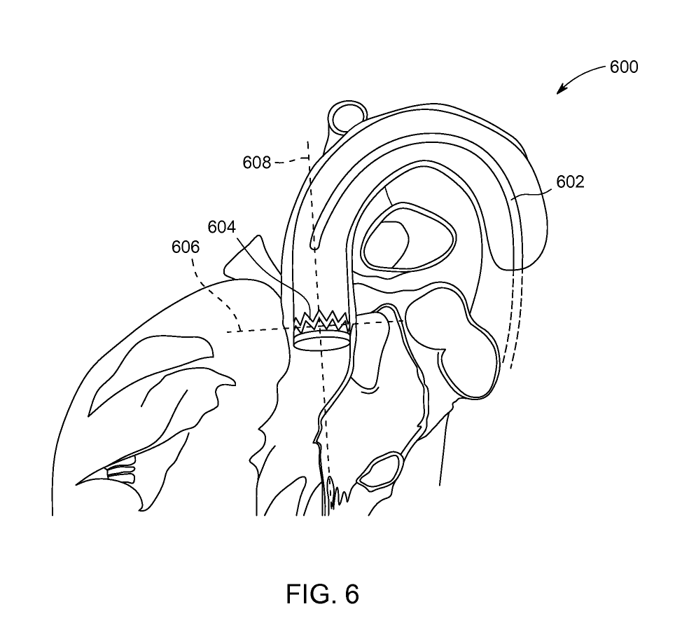

FIG. 6 is a schematic representation of a heart 600 in accordance with an exemplary embodiment. The image of the heart 600 includes a catheter 602, an artificial valve 604, a valve plane 606, and a guideline 608 representing an intended insertion path for the catheter 602 in order to correctly position the artificial valve 604. Those skilled in the art should appreciate that the image of the heart 600 shown in FIG. 6 is a schematic representation showing an exemplary procedure that may be performed using the previously described method 200 shown in FIG. 2. It should be appreciated that other embodiments may show different anatomical structures and that other embodiments may be used to place medical devices other than artificial valves.

The method 200 was described according to an exemplary embodiment using real-time 3D ultrasound data and a live ultrasound image. This exemplary embodiment advantageously provides the user with real-time information regarding the position of the catheter with respect to an intended insertion path. However, it should be appreciated that other embodiments may use 3D ultrasound data that is not real-time 3D ultrasound data. For example, the 3D ultrasound data may be accessed from a memory or other storage device. Additionally or alternatively, the ultrasound image that is displayed may not be a live ultrasound image. For example, the ultrasound image may be updated at a less than real-time rate according to some embodiments.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims or if they include equivalent structural elements with insubstantial differences from the literal language of the claims.

We claim:

1. An ultrasound-based method for guiding a catheter during an interventional procedure:
acquiring 3D ultrasound data;
identifying a reference location based on the 3D ultrasound data;
displaying ultrasound image based on the 3D ultrasound data;
displaying a guideline superimposed on the ultrasound image, where the guideline represents an intended insertion path for the catheter with respect to the reference location; and
inserting the catheter during the process of both acquiring the 3D ultrasound data and displaying the guideline superimposed on the ultrasound image.

2. The method of claim 1, wherein the 3D ultrasound data comprises real-time 3D ultrasound data, and wherein the ultrasound image comprises a live ultrasound image.

3. The method of claim 2, further comprising automatically detecting that the catheter is exceeds a predetermined distance from the intended insertion path and providing feedback to indicate that the catheter is outside of the predetermined distance from the intended insertion path.

4. The method of claim 2, further comprising automatically detecting that the catheter is within a predetermined distance from the intended insertion path and providing feedback to indicate that the catheter is within the predetermined distance from the intended insertion path.

5. The method of claim 2, further comprising automatically tracking a position and an orientation of the reference location in the live ultrasound image during the process of inserting the catheter.

6. The method of claim 5, further comprising adjusting the position of the guideline in real-time in response to said tracking the position and orientation of the reference location to maintain a fixed relationship between the guideline and the reference location.

7. The method of claim 3, wherein the feedback comprises audible feedback.

8. The method of claim 3, wherein the feedback comprises visual feedback.

9. The method of claim 1, wherein the reference location comprises a valve plane and the medical device comprises a replacement valve.

10. The method of claim 9, wherein the guideline is positioned perpendicular to the valve plane.

11. The method of claim 1, wherein said identifying the reference location comprises manually identifying a plurality of points or a contour on the ultrasound image.

12. The method of claim 1, wherein said identifying the reference location comprises automatically detecting an anatomical structure with a border-detection algorithm.

13. The method of claim 2, further comprising detecting a position of the catheter based on an electromagnetic tracking device connected to the catheter, and using the detected position of the catheter to calculate whether the catheter is within the predetermined distance of the intended insertion path during the process of inserting the medical device.

14. An ultrasound imaging system comprising:
a probe;
a display device; and
a processor in electronic communication with the probe and the display device, wherein the processor is configured to:
control the probe to acquire 3D ultrasound data;
display an ultrasound image based on the 3D ultrasound data on the display device;
identify a reference location in the 3D ultrasound data;
display a guideline superimposed on the ultrasound image, where the guideline represents an intended insertion path for a catheter with respect to the reference location;
automatically detect a position of the catheter based on the 3D ultrasound data; and
automatically provide feedback indicating whether the catheter is within a predetermined distance from the intended insertion path during the process of inserting the catheter.

15. The ultrasound imaging system of claim 14, wherein the 3D ultrasound data comprises real-time 3D ultrasound data, and wherein the ultrasound image comprises a live ultrasound image.

16. The ultrasound imaging system of claim 14, wherein the processor is configured to automatically identify the reference location based on an image processing technique.

17. The ultrasound imaging system of claim 14, further comprising a speaker and wherein the feedback comprises audible feedback played through the speaker.

18. The ultrasound imaging system of claim 15, wherein the feedback comprises visual feedback displayed on the display device in real-time.

19. The ultrasound imaging system of claim 15, wherein the processor is configured to track a position and an orientation of the reference location in real-time based on the real-time 3D ultrasound data.

20. The ultrasound imaging system of claim 19, wherein the processor is configured to adjust a position of the guideline in real-time based on the tracked position and orientation of the reference location to maintain a fixed relative position between the guideline and the reference location.

21. The ultrasound imaging system of claim 14, wherein the processor is configured to automatically detect the position of the catheter based on an image processing technique.
